(19) United States
(12) Patent Application Publication
(10) Pub. No.: US 2007/0213296 A1
(43) Pub. Date: Sep. 13, 2007

(54) Compositions and methods for the treatment of immunoinflammatory disorders

(76) Inventor: Yanzhen Zhang, Sudbury, MA (US)

Correspondence Address: CLARK & ELBING LLP, 101 FEDERAL STREET, BOSTON, MA, 02110, US

(21) Appl. No.: 11/714,967

(22) Filed: Mar. 6, 2007

Related U.S. Application Data
(60) Provisional application No. 60/780,028, filed on Mar. 7, 2006.

(51) Int. Cl. A61K 31/7076; A61K 31/573; A61K 31/366; A61K 31/522; A61K 31/59

(52) U.S. Cl. 514/47; 514/171; 514/167; 514/263.34; 514/559; 514/725; 514/166; 514/454

(57) ABSTRACT

The invention features methods and kits for treating an immunoinflammatory disorder, by administering to a patient diagnosed with or at risk of developing such immunoinflammatory disorder an adenosine activity upregulator in combination with one or more additional agents.

### CROSS-REFERENCE TO RELATED APPLICATIONS

This application claims benefit from U.S. Provisional Application No. 60/780,028, filed Mar. 7, 2006, which is hereby incorporated by reference.

### BACKGROUND OF THE INVENTION

This invention relates to the treatment of immunoinflammatory disorders.

Immunoinflammatory conditions are characterized by the inappropriate activation of the body's immune defenses. Rather than targeting infectious invaders, the immune response targets and damages the body's own tissues or transplanted tissues. The tissue targeted by the immune system varies with the disorder. For example, in multiple sclerosis, the immune response is directed against the neuronal tissue, while in Crohn's disease the digestive tract is targeted.

Immunoinflammatory disorders affect millions of individuals and include conditions such as asthma, allergic intraocular inflammatory diseases, arthritis, atopic dermatitis, atopic eczema, diabetes, hemolytic anaemia, inflammatory dermatoses, inflammatory bowel or gastrointestinal disorders (e.g., Crohn's disease and ulcerative colitis), multiple sclerosis, myasthenia gravis, pruritis/inflammation, psoriasis, rheumatoid arthritis, cirrhosis, and systemic lupus erythematosus.

Current treatment regimens for immunoinflammatory disorders, transplanted organ rejection, and graft versus host disease typically rely on immunosuppressive agents. Steroids are known powerful anti-inflammatory agents. However, chronic administration of anti-inflammatory doses of steroids is also limited by well-known toxicities, and the effectiveness of these agents can vary and their use is often accompanied by adverse side effects. For example, prolonged use of steroids has been associated with osteoporosis, high blood pressure, neurological complications, suboptimal immune response, and ocular disturbances, limiting their utility in therapeutic situations. Thus, improved therapeutic agents and methods for the treatment of immunoinflammatory conditions are needed.

There is a need for additional agents for the treatment of immunoinflammatory disorders.

### SUMMARY OF THE INVENTION

The invention generally features methods, and kits for treating immunoinflammatory disorders by administering to a patient in need thereof an adenosine activity upregulator in combination with a corticosteroid, or any of a number of other companion compounds.

In one aspect, the invention features a method for treating an immunoinflammatory disorder by administering to a patient diagnosed with or at risk of developing such a disorder a Group B adenosine activity upregulator in combination with a corticosteroid, an NSAID, or an NsIDI, simultaneously or within fourteen days, ten days, five days, 24 hours, or even 1 hour of each other in amounts sufficient to treat the immunoinflammatory disorder.

In addition, a third drug, e.g., a corticosteroid, an NSAID; a COX-2 inhibitor; a biologic; a small molecule immunomodulator; a DMARD; a xanthine; an NsIDIs, an anticholinergic compounds; a beta receptor agonist; a bronchodilator; a vitamin D analog; a psoralens; a retinoids; or a 5-amino salicylic acid, may be administered to the patient such that the Group B adenosine activity upregulator, the second drug, and the third drug are administered simultaneously or within fourteen days, ten days, five days, or even 24 hours of each other in amounts sufficient to treat the patient.

The invention further features a kit that includes: (i) a composition containing a Group B adenosine activity upregulator and a second drug, e.g., corticosteroid, an NSAID, or an NsIDI, and (ii) instructions for administering the composition to a patient diagnosed with or at risk of developing an immunoinflammatory disorder.

In addition, the invention features a kit that includes: (i) a Group B adenosine activity upregulator (ii) a second drug, e.g., corticosteroid, an NSAID, or an NsIDI, and (iii) instructions for administering the Group B adenosine activity upregulator and the second drug to a patient diagnosed with or at risk of developing an immunoinflammatory disorder.

The invention further features a kit that includes: (i) a drug, e.g., a corticosteroid, corticosteroid, an NSAID, or an NsIDI, and (ii) instructions for administering a Group B adenosine activity upregulator and the drug to a patient diagnosed with or at risk of developing an immunoinflammatory disorder.

The invention additionally features a kit that includes: (i) a Group B adenosine activity upregulator and (ii) instructions for administering the Group B adenosine activity upregulator and a second drug, e.g., a corticosteroid, an NSAID, or an NsIDI, to a patient diagnosed with or at risk of developing an immunoinflammatory disorder.

Combination therapies of the invention are useful for the treatment of immunoinflammatory disorders in combination with other anti-cytokine agents or agents that modulate the immune response to positively effect disease, such as agents that influence cell adhesion, or biologics or small molecules that block the action of IL-6, IL-1, IL-2, IL-12, IL-15 or TNFα (e.g., etanercept, adelimumab, infliximab, or CDP-870). In this example (that of agents blocking the effect of TNFα), the combination therapy reduces the production of cytokines, etanercept or infliximab act on the remaining fraction of inflammatory cytokines, providing enhanced treatment. Examples of small molecule immunomodulators that block cytokines or modulate immune response include agents inhibiting p38 MAP kinase (e.g., doramapimod, SCIO-469, VX-702), ICE (e.g., Pralnacasan) and TACE (e.g., BMS-561392).

In any of the methods, compositions, and kits of the invention, analogs of certain compounds may be employed in lieu of the compounds themselves. Suitable analogs are described herein. Structural analogs of a compound (e.g., prednisoline) or class of compound (e.g., a corticosteroid) do not need to have the same activity as the compound or class to which it is related.

Desirably, the methods, compositions and kits of the invention desirably have increased effectiveness, safety, tolerability, or satisfaction of treatment of a patient suffering from or at risk of suffering from an immunoinflammatory disorder, as compared to methods and compositions using each component of the combination individually.

In particular embodiments of any of the methods of the invention, the Group B adenosine activity upregulator and/or the companion compound may be administered administered simultaneously or within fourteen days, ten days, five days, 24 hours, or even 1 hour of each other in high or low dosages, each of which is defined herein. In particular embodiments of any of the methods of the invention, the Group B adenosine activity upregulator and the second drug may be formulated together as a single composition, or may be formulated and administered separately. When the second drug is a corticosteroid, the Group B adenosine activity upregulator may be administered in any useful dosage, in combination with a useful corticosteroid dosage, e.g., 0.1-1500 mg/day, 0.5-30 mg/day, or 0.5-10 mg/day. The composition may be formulated, for example, for topical or systemic administration. The unit dose form of this formulation can be oral, topical, parenteral, rectal, cutaneous and/or subcutaneous.

In certain embodiments of the compositions, kits, and methods of the invention, the only pharmacologically active agents in the composition or kit, or used in the method, are those recited. In this embodiment, pharmacologically inactive excipients may also be present in the composition.

Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

Unless defined otherwise, all technical and scientific terms used herein have the same meaning as commonly understood to one of ordinary skill in the art. Unless otherwise indicated, such as through context, as used herein, the following terms are intended to have the following meanings in interpreting the present invention.

By “a Group A adenosine activity upregulator” is meant is meant a compound having the formula (I): 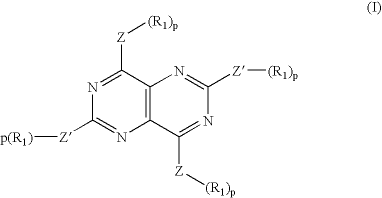
wherein each Z and each Z′ is, independently, N, O, C, 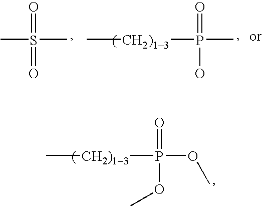
When Z or Z′ is O or 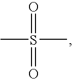
then p=1, when Z or Z′ is N, 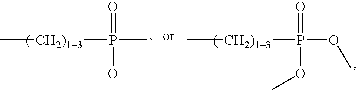
then p=2, and when Z or Z′ is C, then p=3. In formula (I), each R₁ is, independently, X, OH, N-alkyl (wherein the alkyl group has 1 to 20, more preferably 1-5, carbon atoms); a branched or unbranched alkyl group having 1 to 20, more preferably 1-5, carbon atoms; or a heterocycle, as defined herein. Alternatively, when p>1, two R₁ groups from a common Z or Z′ atom, in combination with each other, may represent —(CY₂)ₖ— in which k is an integer between 4 and 6, inclusive. Each X is, independently, Y, CY₃, C(CY₃)₃, CY₂CY₃, (CY₂)₁₋₅OY, substituted or unsubstituted cycloalkane of the structure CₙY₂ₙ₋₁, wherein n=3-7, inclusive. Each Y is, independently, H, F, Cl, Br, or I. In one embodiment, each Z is the same moiety, each Z′ is the same moiety, and Z and Z′ are different moieties.

Examples of Group A adenosine activity upregulators are dipyridamole (also known as 2,6-bis(diethanolamino)-4,8-dipiperidinopyrimido(5,4-d)pyrimidine); 2,6-disubstituted 4,8-dibenzylaminopyrimido[5,4-d]pyrimidines; mopidamole; dipyridamole monoacetate; 2,6-di-(2,2-dimethyl-1,3-dioxolan-4-yl)-methoxy-4,8-di-piperidinopyrimidopyrimidine; 2,6-bis-(2,3-dimethyoxypropoxy)-4,8-di-piperidinopyrimidopyrimidine; 2,6-bis[N,N-di(2-methoxy)ethyl]-4,6-di-piperidinopyrimidopyrimidine, and 2,6-bis(diethanolamino)-4,8-di-4-methoxybenzylaminopyrimidopyrimidine. Other tetra-substituted pyrimidopyrimidines are described in U.S. Pat. Nos. 3,031,450 and 4,963,541, each of which is hereby incorporated by reference.

By “a Group B adenosine activity upregulator” is meant adenosine and any compounds that mimic or potentiate the physiological effects of adenosine and that is not a Group A adenosine activity upregulator. Examples of Group B adenosine activity upregulators include adenosine as well as certain adenosine receptor agonists, adenosine transport inhibitors, adenosine kinase inhibitors, adenylate cyclase stimulants, adenosine deaminase inhibitors, calmodulin antagonists, and phosphodiesterase inhibitors, as described herein.

By “an amount sufficient” is meant the amount of a compound, in a combination of the invention, sufficient to treat or prevent a musculoskeletal disorder or an immunoinflammatory disorder (or pain associated therewith) in a clinically relevant manner. A sufficient amount of an active compound used to practice the present invention for therapeutic treatment of conditions caused by or contributing to the disorder varies depending upon the manner of administration, the age, body weight, and general health of the patient. Ultimately, the prescribers will decide the appropriate amount and dosage regimen. Additionally, “an effective amount” is meant that amount of compound, in a combination of the invention, that is safe and efficacious in the treatment of a patient having the musculoskeletal disorder or an immunoinflammatory disorder over each agent alone as determined and approved by a regulatory authority (such as the U.S. Food and Drug Administration).

By “anticonvulsant” is meant a medication that is used in the prevention of epileptic seizures. Examples of anticonvulsants include carbanazepine, oxcarbazepine, lamotrigine, phenyloin, topiramate, levetivacetam, gabapentin, and valproic acid.

By “corticosteroid” is meant any naturally occurring or synthetic compound characterized by a hydrogenated cyclopentanoperhydrophenanthrene ring system. Naturally occurring corticosteroids are generally produced by the adrenal cortex. Synthetic corticosteroids may be halogenated. Exemplary corticosteroids are described herein.

Corticosteroids useful in the methods, compositions, and kits of the invention include, e.g., algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin. Particularly desirable corticosteroids are prednisolone, cortisone, dexamethasone, hydrocortisone, methylprednisolone, fluticasone, prednisone, triamcinolone, and diflorasone. Desirably, the methods, compositions, and kits of the invention have increased effectiveness, safety, tolerability, or satisfaction of treatment of a patient suffering from or at risk of suffering from a musculoskeletal disorder, or pain associated therewith, as compared to methods and compositions using each component of the combination individually.

By a “high dosage” is meant at least 5% (e.g., at least 10%, 20%, 30%, 40%, 50%, 60%, 70%, 80%, 90%, 100%, 200%, or even 300%) more than the highest standard recommended dosage of a particular compound for treatment of any human disease or condition.

By a “low dosage” is meant at least 5% less (e.g., at least 10%, 20%, 30%, 40%, 50%, 60%, 70%, 80%, 90%, or even 95%) than the lowest standard recommended dosage of a particular compound formulated for a given route of administration for treatment of any human disease or condition. For example, a low dosage of corticosteroid formulated for administration by inhalation will differ from a low dosage of corticosteroid formulated for oral administration.

By a “moderate dosage” is meant the dosage between the low dosage and the high dosage.

By “more effective” is meant that a method, composition, or kit exhibits greater efficacy, is less toxic, safer, more convenient, better tolerated, or less expensive, or provides more treatment satisfaction than another method, composition, or kit with which it is being compared. Efficacy may be measured by a skilled practitioner using any standard method that is appropriate for a given indication.

By “immunoinflammatory disorder” is meant to encompass a variety of conditions, including autoimmune diseases, proliferative skin diseases, and inflammatory dermatoses. Immunoinflammatory disorders result in the destruction of healthy tissue by an inflammatory process, deregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hemolytic anemia; autoimmune hepatitis; Behcet's disease; Bell's palsy; bullous pemphigoid; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; inflammatory bowel or gastrointestinal disorders, inflammatory dermatoses; lichen planus; lupus nephritis; lymphomatous tracheobronchitis; macular edema; multiple sclerosis; myasthenia gravis; myositis; osteoarthritis; pancreatitis; pemphigoid gestationis; pemphigus vulgaris; polyarteritis nodosa; polymyalgia rheumatica; pruritus scroti; pruritis/inflammation, psoriasis; psoriatic arthritis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; tuberculosis; type-1 diabetes; ulcerative colitis; uveitis; vasculitis; and Wegener's granulomatosis. “Non-dermal inflammatory disorders” include, for example, rheumatoid arthritis, inflammatory bowel disease, asthma, and chronic obstructive pulmonary disease.

“Dermal inflammatory disorders” or “inflammatory dermatoses” include, for example, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet's disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

“Non-dermal inflammatory disorders” include, for example, rheumatoid arthritis, inflammatory bowel disease, asthma, and chronic obstructive pulmonary disease.

By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis.

By “musculoskeletal disorder” is meant an immune system-related disorder of the muscles, ligaments, bones, joints, cartilage, or other connective tissue. Among the most commonly occurring musculoskeletal disorders are various forms of arthritis, e.g., osteoarthritis, rheumatoid arthritis, juvenile rheumatoid arthritis, and gout. Other musculoskeletal disorders include acquired hyperostosis syndrome, acromegaly, ankylosing spondylitis, Behcet's disease, bone diseases, bursitis, cartilage diseases, chronic fatigue syndrome, compartment syndromes, congenital hypothyroidism, congenital myopathies, dentigerous cyst, dermatomyositis, diffuse idiopathic skeletal hyperostosis, Dupuytren's contracture, eosinophilia-myalgia syndrome, fasciitis, Felty's syndrome, fibromyalgia, hallux valgus, infectious arthritis, joint diseases, Kabuki make-up syndrome, Legg-Perthes disease, lupus, Lyme disease, Melas syndrome, metabolic bone diseases, mitochondrial myopathies, mixed connective tissue disease, muscular diseases, muscular dystrophies, musculoskeletal abnormalities, musculoskeletal diseases, myositis, myositis ossificans, necrotizing fasciitis, neurogenic arthropathy, osteitis deformans, osteochondritis, osteomalacia, osteomyelitis, osteonecrosis, osteoporosis, Paget's disease, Pierre Robin syndrome, polymyalgia rheumatica, polymyositis, postpoliomyelitis syndrome, pseudogout, psoriatric arthritis, reactive arthritis, Reiter disease, relapsing polychondritis, renal osteodystrophy, rhabdomyolysis, rheumatic diseases, rheumatic fever, scleroderma, Sever's disease (calceneal apophysitis), Sjogren's syndrome, spinal diseases, spinal stenosis, Still's disease, synovitis, temporomandibular joint disorders, tendinopathy, tennis elbow, tenosynovitis, Tietze's syndrome, and Wegener's granulomatosis.

As will be appreciated by one skilled in the art, a particular disease, disorder, or condition may be characterized as being both musculoskeletal and immunoinflammatory. An example of such a disease is osteoarthritis.

The term “pain” is used herein in the broadest sense and refers to all types of pain, including acute and chronic pain, such as nociceptive pain, e.g. somatic pain and visceral pain; neuropathic pain, e.g., centrally generated pain and peripherally generated pain; and psychogenic pain. The term preferably refers to chronic pain, most preferably nociceptive pain, including somatic pain and visceral pain.

The term “nociceptive pain” is used to include all pain caused by injury to body tissues, including, without limitation, by a cut, bruise, bone fracture, crush injury, burn, and the like. This type of pain is typically aching, sharp, or throbbing. Pain receptors for tissue injury (nociceptor) are located mostly in the skin or in the internal organs.

The term “somatic pain” is used to refer to pain arising from bone, joint, muscle, skin, or connective tissue. This type of pain is typically aching or throbbing in quality and is well localized.

The term “visceral pain” is used herein to refer to pain arising from visceral organs, such as the gastrointestinal tract and pancreas. Visceral pain includes aching and fairly well localized pain caused by tumor involvement of the organ capsule. Another type of visceral pain, which is typically caused by obstruction of hollow viscus, is characterized by intermittent cramping and poorly localized pain.

The term “neuropathic pain” is used herein to refer to pain originating from abnormal processing of sensory input by the peripheral or central nervous system.

By “non-steroidal anti-inflammatory drug” or “NSAID” is meant a non-steroidal agent that prevents or diminishes inflammation. NSAIDs include naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, tolmetin, and COX-2 inhibitors such as rofecoxib, celecoxib, valdecoxib, or lumiracoxib.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “opioid” is meant any agent that binds to opioid receptors. Non-limiting examples of opioids include codeine, hydrocodone, morphine, hydromorphone, methadone and fentanyl.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Examplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

By “sustained release” or “controlled release” is meant that the therapeutically active component is released from the formulation at a controlled rate such that therapeutically beneficial blood levels (but below toxic levels) of the component are maintained over an extended period of time ranging from e.g., about 12 to about 24 hours, thus, providing, for example, a 12 hour or a 24 hour dosage form.

By “systemic administration” is meant all nondermal routes of administration, and specifically excludes topical and transdermal routes of administration.

By “treating” is meant administering or prescribing a composition for the treatment or prevention of a musculoskeletal disorder or an immunoinflammatory disorder.

By “tricyclic antidepressant” is meant a chemical compound with a tricyclic ring structure used for the treatment and prevention of depression. Examples of tricyclic antidepressants include amitriptyline, imipramine, desipramine, nortriptyline, and paroxetine.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Other features and advantages of the invention will be apparent from the following detailed description, and from the claims.

### BRIEF DESCRIPTION OF THE DRAWINGS

FIG. 1 is a graph showing suppression of LPS-induced TNFα secretion in cells trearted with rolipram and prednisolone.

FIG. 2 is a graph showing suppression of PMA/ionomycin-induced TNFα secretion in cells trearted with rolipram and prednisolone.

### DETAILED DESCRIPTION

The invention features, methods, compositions, and kits useful for the treatment of musculoskeletal disorders, immunoinflammatory disorders, and pain. According to the invention, a musculoskeletal disorder, immuninflammatory disorder, or associated pain may be treated by administration of an effective amount of an adenosine activity upregulator or analog thereof, in combination with one or more companion compounds, including a corticosteroid, a non-steroidal anti-inflammatory drug (NSAID), or a non-steroidal immunophilin-dependent immunosuppressant (NsIDI), or an analog of any thereof. Furthermore, according to the invention, pain may be treated by administration of an effective amount of an adenosine activity upregulator or analog thereof, in combination with one or more companion compounds, including a corticosteroid, an NSAID, an opioid, a tricyclic antidepressant, an anticonvulsant, amantadine, tramadol, oxycodone, buproprion, mexiletine, or capsaicin, or an analog of any thereof.

In some instances, each component of a combination of the invention may affect only part of a particular disease network, leading to incomplete or no effect on its own, while the combination selectively amplifies one or more therapeutic effects without recapitulating the toxicity of either component alone. For example, the combination of an adenosine activity upregulator and a corticosteroid can result in amplified anti-inflammatory or immunosuppressive effects in comparison to the administration of an effective dose of either agent alone, while resulting in significantly reduced toxicity.

Routes of administration for the various embodiments include, but are not limited to, topical, transdermal, and systemic administration (such as intravenous, intramuscular, subcutaneous, inhalation, rectal, buccal, vaginal, intrathecal, intraperitoneal, intraarticular, ophthalmic, or oral administration). As used herein, “systemic administration” refers to all nondermal routes of administration, and specifically excludes topical and transdermal routes of administration.

Any of the foregoing therapies may be administered with conventional pharmaceuticals useful for the treatment of musculoskeletal disorders, immunoinflammatory disorders, or pain.

The invention is described in more detail below.

Adenosine and Adenosine Activity Upregulators

The endogenous purine nucleoside, adenosine is an extracellular signaling molecule which interacts with a family of extracellular P₁ G-protein coupled receptors (A₁, A_{2A}, A_{2B}, and A₃). Under certain conditions, the local tissue concentrations of extracellular ADO are increased after the release of adenosine itself and/or that of AMP, which is metabolized extracellularly to produce adenosine.

Compounds that mimic the physiological effects of adenosine, such as adenosine receptor agonists, adenosine transport inhibitors, adenosine kinase inhibitors, adenylate cyclase stimulants (e.g., ORG 2766 (Organon), and Colforsin dapropate (Nippon Kayaku, Sanofi-Aventis)), adenosine deaminase inhibitors (e.g., Pentostatin (National Cancer Institute (USA), Pfizer)), calmodulin antagonists (e.g., Zaldaride (Novartis Consumer Health) and Bepridil (RETI)), and phosphodiesterase (PDE) inhibitors, are discussed herein.

Adenosine Receptor Agonists

Examples of adenosine receptor agonists that can be employed in the methods, compositions, and kits of the invention are adenosine hemisulfate salt, adenosine amine congener solid, N⁶-(4-amino-3-iodophenyl)methyl-5′-N-methylcarboxamidoadenosine (I-AB-MECA); N-((2-methylphenyl)methyl)adenosine (Metrifudil); 2-(1-hexynyl)-N-methyladenosine (HEMADO); N-(1-methyl-2-phenylethyl)adenosine (R-PIA); N⁶—(R-4-hydroxyphenylisopropyl) adenosine (HPIA); N⁶-cyclopentyladenosine (CPA); N⁶-cyclopentyl-2-(3-phenylaminocarbonyltriazene-1-yl)adenosine (TCPA); N-((1S,trans)-2-hydroxycyclopentyl)adenosine (GR 79236); N⁶-cyclohexyladenosine (CHA); 2-chloro-N⁶-cyclopentyladenosine (CCPA); N-ethylcarboxamidoadenosine (NECA); 2-(4-(2-carboxyethyl)phenethylamino)-5′-N-ethylcarboxamidoadenosine (CGS 21680); N⁶-(3-iodobenzyl)-5′-N-methylcarboxamidoadenosine (IB-MECA); 2-(cyclohexylmethylidene hydrazino)adenosine (WRC 0470); 2-(4-(2-carboxyethyl)phenethylamino)-5′-N-ethylcarboxamidoadenosine (CGS 21680); N⁶-(2-(3,5-dimethoxyphenyl)-2-(2-methylphenyl)ethyl)adenosine (DPMA); hexynyladenosine-5′-N-ethylcarboxamide (HE-NECA); 2-[(2-aminoethyl-aminocarbonylethyl) phenylethylamino]-5′-N-ethyl-carboxamidoadenosine (APEC); 2-chloro-N-(3-iodobenzyl)-5′-N-methylcarboxamidoadenosine (2-Cl-IB-MECA); 2-phenylaminoadenosine (CV 1808); 3′-Aminoadenosine-5′-uronamides; CV Therapuetics™ small molecule drugs Tecadenoson (CVT-510); Regadenoson (CVT 3146); and Carisa (CVT 3033); and Aderis Pharmaceuticals™ small drug molecules 2-[2-(4-chlorophenyl)ethoxy]adenosine (MRE 0094), 1-deoxy-1-[6-[[(iodophenyl)methyl]amino]-9H-purine-9-yl]-N-methyl-(-D-ribofuranuronamide) (CF101), Selodenoson (DTI-0009), Apadenoson (University of Virginia), and Binodenoson (MRE-0470). Other adenosine receptor agonists are those described or claimed in Gao et al., JPET, 298: 209-218 (2001); U.S. Pat. Nos. 5,278,150, 5,877,180, 6,232,297; U.S. Patent Application Publication No. 20050261236, and PCT Publication No. WO/9808855, incorporated herein by reference.

Adenosine Transport Inhibitors

Adenosine transport inhibitors that can be employed in the methods, compositions, and kits of the invention include 3-[1-(6,7-diethoxy-2-morpholinoquinazolin-4-yl)piperidin-4-yl]-1,6-dimethyl-2,4(1H,3H)-quinazolinedione hydrochloride (KF24345); 6-(4-nitrobenzyl)-thioinosine (NBI) and 6-(2-hydroxy-5-nitrobenzyl)-thioguanosine (NBG); 6-[4-(1-cyclohexyl-1H-tetrazol-5-yl)butoxy]-3,4-dihydro-2(1H)-quinolinone (Cilostazol); (2-amino-4,5-dimethyl-3-thienyl)-[3-(trifluoromethyl) phenyl]methanone PD 81723); 3,7-dihydro-3-methyl-1-(5-oxohexyl)-7-propyl-1H-purine-2,6-dione (propentofylline); 6-[(4-nitrobenzyl)thio]-9-β-D-ribofuranosylpurine (nitrobenzylthioinosine) (NBMR); 3,4,5-trimethoxy-, (tetrahydro-1H-1,4-diazepine-1,4(5H)-diyl)di-3,1-propanediyl benzoic acid, ester (dilazep); hexobendine; dipyridamole; and adenosine transport inhibitors described in Fredholm, J. Neurochem. 62: 563-573 (1994), Noji et al., J. Pharmacol. Exp. Ther. 300: 200-205 (2002); and Crawley et al.; Neurosci Lett. 36: 169-174 (1983), each of which is incorporated herein by reference.

Adenosine Kinase Inhibitors

Adenosine kinase inhibitors can be used as adenosine activity upregulators in the methods, compositions, and kits of the invention. Adenosine kinase inhibitors are generally described as either nucleoside-like, or nonnucleoside-like.

Nucleoside-like Adenosine Kinase Inhibitors

Nucleoside-like adenosine kinase inhibitors that can be used in the methods, compositions, and kits of the invention include 5-iodotubercidin (5IT) and 2-diaryltubercidin analogues; 5′-deoxo-5′-deoxy-5-iodotubercidin (5′d-5IT); and 5′-deoxo-5′-aminoadenosine (NH₂dADO). Other nucleoside-like adenosine kinase inhibitors are described in McGaraughty et al., Current Topics in Medicinal Chemistry 5: 43-58 (2005); Ugarkar, J. Med. Chem. 43: 2883-2893 (2000); Ugarkar et al., J. Med. Chem. 43: 2894-2905 (2000); Kaplan and Coyle, Eur. J. Pharmacol. 1: 1-8 (1998); and Sinclair et al. Br. J. Pharmacol. 5: 1037-1044 (2001), each of which is incorporated herein by reference.

Nonnucleoside-Like Adenosine Kinase Inhibitors

Nonnucleoside-like adenosine kinase inhibitors that can be used in the methods, compositions, and kits of the invention include 5-bromopyrrolopyrrolidine; 4-amino-5-(3-bromophenyl)-7-(6-morpholino-pyridin-3-yl)pyrido[2,3-d]pyrimidine (ABT-702). Other nonnucleoside-like AK inhibitors are described in McGaraughty et al., Current Topics in Medicinal Chemistry 5: 43-58 (2005), Gomtsyan and Lee, Current Pharmaceutical Design 10: 1093-1103 (2004); Jarvis et al. J. Pharm. Exp. Ther. 295: 1156-1164 (2000); Kowaluk, et al. J. Pharm. Exp. Ther. 295: 1165-1174 (2000); and German Patent Application DE 1014 A1, each of which is incorporated herein by reference.

Phosphodiesterase Inhibitors

Several isozymes of phosphodiesterases act as regulatory switches by catalyzing the degradation of cAMP to adenosine-5-monophosphate (5′-AMP). Inhibitors of phosphodiesterases can lead to an increase in cAMP levels, which in turn can lead to an increase in antiinflammatory actions.

Type I Phosphodiesterase Inhibitors

Type I PDE inhibitors that can be employed in the methods, compositions, and kits of the invention include (3-alpha, 16-alpha)-eburnamenine-14-carboxylic acid ethyl ester (Vinpocetine); 18-methoxymethyl-3-isobutyl-1-methylxantine (MIMX); 1-carboxy-2,3,4,4a,4b,5,6,6a,6b,7,8,8a,8b,9,10,10a,14,16,17,17a,17b,18,19,19a,19b, 20,21,21a,21b,22,23,23a-dotriacontahydro-14-hydroxy-8a, 10a-bis(hydroxymethyl)-14-(3-methoxy-3-oxopropyl)-1,4,4a,6,6a,17b,19b,21b-octamethyl beta-D-glucopyranosiduronic acid (Ks-505a); cis-5,6a,7,8,9,9a-hexahydro-2-(4-(trifluoromethyl)phenylmethyl)-5-methyl-cyclopent (4,5)imidazo[2,1-b]purin-4(3H)-one (SCH 51866); and 2-o-propoxyphenyl-8-azapurine-6-one (Zaprinast). Other Type I PDE inhibitors are described in U.S. Patent Application Nos. 20040259792 and 20050075795, incorporated herein by reference.

Type II Phosphodiesterase Inhibitors

Type II PDE inhibitors that can be employed in the methods, compositions, and kits of the invention include erythro-9-(2-hydroxy-3-nonyl)adenine (EHNA); 2,3,6,7-tetrahydro-9,10-dimethoxy-3-methyl-2-((2,4,6-trimethylphenyl)imino)-4H-pyrimido(6,1-a)isoquinolin-4-one (trequinsin); ND7001 (Neuro3D Pharmaceuticals); and BAY 60-7550 (Alexis Biochemicals). Other Type II PDE inhibitors are described in U.S. Patent Application No. 20030176316, incorporated herein by reference.

Type III Phosphodiesterase Inhibitors

Type III PDE inhibitors that can be employed in the methods, compositions, and kits of the invention include 3-isobutyl-1-methylxanthine (IBMX); 6-dihydro-2-methyl-6-oxo-3,4′-bipyridine)-5-carbonitrile (milrinone) and N-cyclohexyl-4-((1,2-dihydro-2-oxo-6-quinolinyl)oxy)-N-methyl-butanamide (cilostamide). Other Type III PDE inhibitors are described in the following patents and patent applications: EP 0 653 426, EP 0 294 647, EP 0 357 788, EP 0 220 044, EP 0 326 307, EP 0 207 500, EP 0 406 958, EP 0 150 937, EP 0 075 463, EP 0 272 914, and EP 0 112 987, U.S. Pat. Nos. 4,963,561; 5,141,931, 6,897,229, and 6,156,753; U.S. Patent Application Nos. 20030158133, 20040097593, 20060030611, and 20060025463; WO 96/15117; DE 2825048; DE 2727481; DE 2847621; DE 3044568; DE 2837161; and DE 3021792, each of which is incorporated herein by reference.

Type IV Phosphodiesterase Inhibitors

Type IV PDE inhibitors that can be employed in the methods, compositions, and kits of the invention include 4-(3-cyclopentyloxy-4-methoxyphenyl)-2-pyrrolidone (rolipram) and 4-(3-butoxy-4-methoxybenzyl)-2-imidazolidinone (Ro20-1724), Cilomilast (GlaxoSmithKline), Rolipram (Schering AG), MN 001 (Kyorin Pharmaceutical), Arofylline (Almirall-Prodesfarma), Tofimilast (Pfizer), Oglemilast (Glenmark Pharmaceuticals Ltd), Tetomilast (Otsuka Pharmaceutical), and Roflumilast (ALTANA Pharma). Other Type IV PDE inhibitors are described in the following patents, patent applications, and references: U.S. Pat. Nos. 3,892,777, 4,193,926, 4,655,074, 4,965,271, 5,096,906, 5,124,455, 5,272,153, 6,569,890, 6,953,853, 6,933,296, 6,919,353, 6,953,810, 6,949,573, 6,909,002, and 6,740,655; U.S. Patent Application Nos. 20030187052, 20030187257, 20030144300, 20030130254, 20030186974, 20030220352, 20030134876, 20040048903, 20040023945, 20040044036, 20040106641, 20040097593, 20040242643, 20040192701, 20040224971, 20040220183, 20040180900, 20040171798, 20040167199, 20040146561, 20040152754, 20040229918, 20050192336, 20050267196, 20050049258, 20060014782, 20060004003, 20060019932, 20050267196, 20050222207, 20050222207, 20060009481; PCT Publication No. WO 92/079778; and Molnar-Kimber, K. L. et al. J. Immunol, 150: 295 A (1993), each of which is incorporated herein by reference.

Type V Phosphodiesterase Inhibitors

Type V PDE inhibitors that can be used in the methods, compositions, and kits of the invention include those described in U.S. Pat. Nos. 6,992,192, 6,984,641, 6,960,587, 6,943,166, 6,878,711, and 6,869,950, and U.S. Patent Application Nos. 20030144296, 20030171384, 20040029891, 20040038996, 20040186046, 20040259792, 20040087561, 20050054660, 20050042177, 20050245544, 20060009481, each of which is incorporated herein by reference.

Type VI Phosphodiesterase Inhibitors

Type VI PDE inhibitors that can be used in the methods, compositions, and kits of the invention include those described in U.S. Patent Application Nos. 20040259792, 20040248957, 20040242673, and 20040259880, each of which is incorporated herein by reference.

Type VII Phosphodiesterase Inhibitors

Type VII PDE inhibitors that can be used in the methods, compositions, and kits of the invention include those described in the following patents, patent application, and references: U.S. Pat. Nos. 6,838,559, 6,753,340, 6,617,357, and 6,852,720; U.S. Patent Application Nos. 20030186988, 20030162802, 20030191167, 20040214843, and 20060009481; PCT Publication WO 00/68230; and Martinez et al., J. Med. Chem. 43: 683-689 (2000), each of which is incorporated herein by reference.

Corticosteroids

Suitable corticosteroids include those from the class of selective glucocorticosteroid receptor agonists (SEGRAs), 11-alpha, 17-alpha,21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11) -triene-3,20-dione; 17-alpha-hydroxypregn-4-ene-3,20-dione; 17-alpha-hydroxypregnenolone; 17-hydroxy-16-beta-methyl-5-beta-pregn-9(11) -ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

Standard recommended dosages for various steroid/disease combinations are provided in Table 1, below.

TABLE 1
Standard Recommended Corticosteroid Dosages

| Indication | Route | Drug | Dose | | Schedule |
|---|---|---|---|---|---|
| Psoriasis | oral | prednisolone | 7.5-60 | mg | per day or divided b.i.d. |
| | oral | prednisone | 7.5-60 | mg | per day or divided b.i.d. |
| Asthma | inhaled | beclomethasone dipropionate | 42 | μg/puff) | 4-8 puffs b.i.d. |
| | inhaled | budesonide | (200 | μg/inhalation) | 1-2 inhalations b.i.d. |
| | inhaled | flunisolide | (250 | μg/puff) | 2-4 puffs b.i.d. |
| | inhaled | fluticasone propionate | (44, 110 or 220 | μg/puff) | 2-4 puffs b.i.d. |
| | inhaled | triamcinolone acetonide | (100 | μg/puff) | 2-4 puffs b.i.d. |
| COPD | oral | prednisone | 30-40 | mg | per day |
| Crohn's disease | oral | budesonide | 9 | mg | per day |
| Ulcerative colitis | oral | prednisone | 40-60 | mg | per day |
| | oral | hydrocortisone | 300 | mg (IV) | per day |
| | oral | methylprednisolone | 40-60 | mg | per day |
| Rheumatoid arthritis | oral | prednisone | 10 | mg | per day |

Other standard recommended dosages for corticosteroids are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone. When the combinations of the invention are used for treatment in conjunction with corticosteroids, it is possible to reduce the dosage of the individual components substantially to a point significantly below the dosages which would be required to achieve the same effects by administering corticosteroids or an adenosine activity upregulator alone or by administering a combination of corticosteroids and an adenosine activity upregulator. For example, in an adenosine activity upregulator/corticosteroid combination, reduced dosages of the adenosine activity upregulator or the corticosteroid, in comparison with dosages appropriate for administration of either compound alone, may be effective in treating a musculoskeletal disorder and/or immunoinflammatory disorder or pain associated therewith. Two or more corticosteroids can be administered in the same treatment.

Steroid Receptor Modulators

Steroid receptor modulators (e.g., antagonists and agonists) may be used as a substitute for or in addition to a corticosteroid in the methods, compositions, and kits of the invention. Thus, in one embodiment, the invention features the combination of an adenosine activity upregulator and a glucocorticoid receptor modulator or other steroid receptor modulator, and methods of treating musculoskeletal disorders and/or immunoinflammatory disorders, or pain associated with such disorders, therewith.

Glucocorticoid receptor modulators that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used as a substitute for or in addition to a corticosteroid in the methods, compositions, and kits of the invention include A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), RU24858, RU40066, AL-438, ZK216348, NCX-1004, A276575, and ZK-73634 (Schering AG).

Non-Steroidal Anti-Inflammatory Drugs

If desired, the adenosine activity upregulator may be administered in conjunction with one or more of non-steroidal anti-inflammatory drugs (NSAIDs), such as acetoaminophen, naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin.

Acetylsalicylic acid, also known by trade name aspirin, is an acetyl derivative of salicylic acid. Aspirin is useful in the relief of headache and muscle and joint aches. Aspirin is also effective in reducing fever, inflammation, and swelling and thus has been used for treatment of rheumatoid arthritis, rheumatic fever, and mild infection. Thus in one aspect, combination of an adenosine activity upregulator and acetylsalicylic acid (aspirin) or analog thereof can also be administered to enhance the treatment or prevention of the diseases mentioned above.

An NSAID may be administered in conjunction with any one of the combinations described in this application. For example, a patient suffering from a musculoskeletal or immunoinflammatory disorder may be initially treated with a combination of an adenosine activity upregulator/glucocorticoid receptor modulator combination and then the patient may also be treated with an NSAID, such as acetylsalicylic acid, in conjunction with the combinations described above.

Dosage amounts of acetylsalicylic acid are known to those skilled in medical arts, and generally range from about 70 mg to about 350 mg per day. When a lower or a higher dose of aspirin is needed, a formulation containing dipyridamole and aspirin may contain 0-25 mg, 25-50 mg, 50-70 mg, 70-75 mg, 75-80 mg, 80-85 mg, 85-90 mg, 90-95 mg, 95-100 mg, 100-150 mg, 150-160 mg, 160-250 mg, 250-300 mg, 300-350 mg, or 350-1000 mg of aspirin.

Nonsteroidal Immunophilin-Dependent Immunosuppressants

In healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus), and rapamycin target many types of immunoregulatory cells, including T-cells, and suppress the immune response in organ transplantation and autoimmune disorders.

In one embodiment, the NsIDI is cyclosporine, and is administered in an amount between 0.05 and 50 milligrams per kilogram per day (e.g., orally in an amount between 0.1 and 12 milligrams per kilogram per day). In another embodiment, the NsIDI is tacrolimus and is administered in an amount between 0.0001-20 milligrams per kilogram per day (e.g., orally in an amount between 0.01-0.2 milligrams per kilogram per day). In another embodiment, the NsIDI is rapamycin and is administered in an amount between 0.1-502 milligrams per day (e.g., at a single loading dose of 6 mg/day, followed by a 2 mg/day maintenance dose). In another embodiment, the NsIDI is everolimus, administered at a dosage of 0.75-8 mg/day. In still other embodiments, the NsIDI is pimecrolimus, administered in an amount between 0.1 and 200 milligrams per day (e.g., as a 1% cream/twice a day to treat atopic dermatitis or 60 mg a day for the treatment of psoriasis), or the NsIDI is a calcineurin-binding peptide administered in an amount and frequency sufficient to treat the patient. Two or more NsIDIs can be administered contemporaneously.

Cyclosporines

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70: 365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al. (Antimicrob. Agents Chemother. 44: 143-149, 2000). Cyclosporines are highly hydrophobic and readily precipitate in the presence of water (e.g. on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

Cyclosporines can be administered either intravenously or orally, but oral administration is preferred. To overcome the hydrophobicity of cyclosporine A, an intravenous cyclosporine A is usually provided in an ethanol-polyoxyethylated castor oil vehicle that must be diluted prior to administration. Cyclosporine A may be provided, e.g., as a microemulsion in a 25 mg or 100 mg tablets, or in a 100 mg/ml oral solution (NEORAL).

Typically, patient dosage of an oral cyclosporine varies according to the patient's condition, but some standard recommended dosages are provided herein. Patients undergoing organ transplant typically receive an initial dose of oral cyclosporine A in amounts between 12 and 15 mg/kg/day. Dosage is then gradually decreased by 5% per week until a 7-12 mg/kg/day maintenance dose is reached. For intravenous administration 2-6 mg/kg/day is preferred for most patients. For patients diagnosed as having Crohn's disease or ulcerative colitis, dosage amounts from 6-8 mg/kg/day are generally given. For patients diagnosed as having systemic lupus erythematosus, dosage amounts from 2.2-6.0 mg/kg/day are generally given. For psoriasis or rheumatoid arthritis, dosage amounts from 0.5-4 mg/kg/day are typical. A suggested dosing schedule is shown in Table 2. Other useful dosages include 0.5-5 mg/kg/day, 5-10 mg/kg/day, 10-15 mg/kg/day, 15-20 mg/kg/day, or 20-25 mg/kg/day. Often cyclosporines are administered in combination with other immunosuppressive agents, such as glucocorticoids.

TABLE 2

| | Atopic | | | | | | |
|---|---|---|---|---|---|---|---|
| Compound | Dermatitis | Psoriasis | RA | Crohn's | UC | Transplant | SLE |
| CsA | N/A | 0.5-4 | 0.5-4 | 6-8 | 6-8 | ˜7-12 | 2.2-6.0 |
| (NEORAL) | | mg/kg/day | mg/kg/day | mg/kg/day | mg/kg/day | mg/kg/day | mg/kg/day |
| | | | | (oral- | (oral) | | |
| | | | | fistulizing) | | | |
| Tacrolimus | 0.03-0.1% | 0.05-1.15 | 1-3 | 0.1-0.2 | 0.1-0.2 | 0.1-0.2 | N/A |
| | cream/twice | mg/kg/day | mg/day | mg/kg/day | mg/kg/day | mg/kg/day | |
| | day (30 and | (oral) | (oral) | (oral) | (oral) | (oral) | |
| | 60 gram | | | | | | |
| | tubes) | | | | | | |
| Pimecrolimus | 1% | 40-60 | 40-60 | 80-160 | 160-240 | 40-120 | 40-120 |
| | cream/twice | mg/day | mg/day | mg/day | mg/day | mg/day | mg/day |
| | day (15, 30, | (oral) | (oral) | (oral) | (oral) | (oral) | (oral) |
| | 100 gram | | | | | | |
| | tubes) | | | | | | |

Table Legend
CsA = cyclosporine A
RA = rheumatoid arthritis
UC = ulcerative colitis
SLE = systemic lupus erythamatosus

Tacrolimus

Tacrolimus (FK506) is an immunosuppressive agent that targets T cell intracellular signal transduction pathways. Tacrolimus binds to an intracellular protein FK506 binding protein (FKBP-12) that is not structurally related to cyclophilin (Harding et al. Nature 341: 758-7601, 1989; Siekienka et al. Nature 341: 755-757, 1989; and Soltoff et al., J. Biol. Chem. 267: 17472-17477, 1992). The FKBP/FK506 complex binds to calcineurin and inhibits calcineurin's phosphatase activity. This inhibition prevents the dephosphorylation and nuclear translocation of nuclear factor of activated T cells (NFAT), a nuclear component that initiates gene transcription required for proinflammatory cytokine (e.g., IL-2, gamma interferon) production and T cell activation. Thus, tacrolimus inhibits T cell activation.

Tacrolimus is a macrolide antibiotic that is produced by Streptomyces tsukubaensis. It suppresses the immune system and prolongs the survival of transplanted organs. It is currently available in oral and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 0.9% sodium chloride or 5% dextrose prior to injection. While oral administration is preferred, patients unable to take oral capsules may receive injectable tacrolimus. The initial dose should be administered no sooner than six hours after transplant by continuous intravenous infusion.

Tacrolimus and tacrolimus analogs are described by Tanaka et al., (J. Am. Chem. Soc., 109: 5031, 1987) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

While suggested dosages will vary with a patient's condition, standard recommended dosages are provided below. Typically patients diagnosed as having Crohn's disease or ulcerative colitis are administered 0.1-0.2 mg/kg/day oral tacrolimus. Patients having a transplanted organ typically receive doses of 0.1-0.2 mg/kg/day of oral tacrolimus. Patients being treated for rheumatoid arthritis typically receive 1-3 mg/day oral tacrolimus. For the treatment of psoriasis, 0.01-0.15 mg/kg/day of oral tacrolimus is administered to a patient. Atopic dermatitis can be treated twice a day by applying a cream having 0.03-0.1% tacrolimus to the affected area. Patients receiving oral tacrolimus capsules typically receive the first dose no sooner than six hours after transplant, or eight to twelve hours after intravenous tacrolimus infusion was discontinued. Other suggested tacrolimus dosages include 0.005-0.01 mg/kg/day, 0.01-0.03 mg/kg/day, 0.03-0.05 mg/kg/day, 0.05-0.07 mg/kg/day, 0.07-0.10 mg/kg/day, 0.10-0.25 mg/kg/day, or 0.25-0.5 mg/kg/day.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus

Pimecrolimus is the 33-epi-chloro derivative of the macrolactam ascomyin. Pimecrolimus structural and functional analogs are described in U.S. Pat. No. 6,384,073. Pimecrolimus is particularly useful for the treatment of atopic dermatitis. Pimecrolimus is currently available as a 1% cream. Suggested dosing schedule for pimecrolimus is shown at Table 2. While individual dosing will vary with the patient's condition, some standard recommended dosages are provided below. Oral pimecrolimus can be given for the treatment of psoriasis or rheumatoid arthritis in amounts of 40-60 mg/day. For the treatment of Crohn's disease or ulcerative colitis amounts of 80-160 mg/day pimecrolimus can be given. Patients having an organ transplant can be administered 160-240 mg/day of pimecrolimus. Patients diagnosed as having systemic lupus erythamatosus can be administered 40-120 mg/day of pimecrolimus. Other useful dosages of pimecrolimus include 0.5-5 mg/day, 5-10 mg/day, 10-30 mg/day, 40-80 mg/day, 80-120 mg/day, or even 120-200 mg/day.

Rapamycin

Rapamycin is a cyclic lactone produced by Streptomyces hygroscopicus. Rapamycin is an immunosuppressive agent that inhibits T cell activation and proliferation. Like cyclosporines and tacrolimus, rapamycin forms a complex with the immunophilin FKBP-12, but the rapamycin-FKBP-12 complex does not inhibit calcineurin phosphatase activity. The rapamycin immunophilin complex binds to and inhibits the mammalian kinase target of rapamycin (mTOR). mTOR is a kinase that is required for cell-cycle progression. Inhibition of mTOR kinase activity blocks T cell activation and proinflammatory cytokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Rapamycin is currently available for oral administration in liquid and tablet formulations. RAPAMUNE liquid contains 1 mg/mL rapamycin that is diluted in water or orange juice prior to administration. Tablets containing 1 or 2 mg of rapamycin are also available. Rapamycin is preferably given once daily as soon as possible after transplantation. It is absorbed rapidly and completely after oral administration. Typically, patient dosage of rapamycin varies according to the patient's condition, but some standard recommended dosages are provided below. The initial loading dose for rapamycin is 6 mg. Subsequent maintenance doses of 0.5-2 mg/day are typical. Alternatively, a loading dose of 3 mg, 5 mg, 10 mg, 15 mg, 20 mg, or 25 mg can be used with a 1 mg, 3 mg, 5 mg, 7 mg, or 10 mg per day maintenance dose. In patients weighing less than 40 kg, rapamycin dosages are typically adjusted based on body surface area; generally a 3 mg/m² day loading dose and a 1 mg/m²/day maintenance dose is used.

Peptide Moieties

Peptides, peptide mimetics, peptide fragments, either natural, synthetic or chemically modified, that impair the calcineurin-mediated dephosphorylation and nuclear translocation of NFAT are suitable for use in practicing the invention. Examples of peptides that act as calcineurin inhibitors by inhibiting the NFAT activation and the NFAT transcription factor are described, e.g., by Aramburu et al., Science 285: 2129-2133, 1999) and Aramburu et al., Mol. Cell 1: 627-637, 1998). As a class of calcineurin inhibitors, these agents are useful in the methods of the invention.

Therapy

Therapy according to the invention may be performed alone or in conjunction with another therapy and may be provided at home, the doctor's office, a clinic, a hospital's outpatient department, or a hospital. The duration of the therapy depends on the type of disease or disorder being treated, the age and condition of the patient, the stage and type of the patient's disease, and how the patient responds to the treatment. Additionally, a person having a greater risk of developing an inflammatory disease (e.g., a person who is undergoing age-related hormonal changes) may receive treatment to inhibit or delay the onset of symptoms.

In particular embodiments of any of the methods of the invention, the compounds are administered simultaneously or within fourteen days, ten days, five days, 24 hours, or 1 hour of each other in amounts sufficient to treat the patient. The compounds may be formulated together as a single composition, or may be formulated and administered separately. One or both compounds may be administered in a low dosage or in a high dosage, each of which is defined herein. It may be desirable to administer to the patient other compounds, such as a corticosteroid, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), NsIDIs (e.g., cyclosporine, tacrolimus, pimecrolimus, and ISAtx247), or analogs thereof. Combination therapies of the invention are especially useful for the treatment of immunoinflammatory disorders in combination with other agents that modulate the immune response to positively affect disease. Such agents include those that deplete key inflammatory cells, influence cell adhesion, or influence cytokines involved in immune response. This last category includes both agents that mimic or increase the action of anti-inflammatory cytokines such as IL-10, as well as agents inhibit the activity of pro-inflammatory cytokines such as IL-6, IL-1, IL-2, IL-12, IL-15 or TNFα. Agents that inhibit TNFα include etanercept, adelimumab, infliximab, and CDP-870. In this example (that of agents blocking the effect of TNFα), the combination therapy reduces the production of cytokines, etanercept or infliximab act on the remaining fraction of inflammatory cytokines, providing enhanced treatment. Small molecule immunodulators include, e.g., p38 MAP kinase inhibitors such as VX 702, SCIO 469, doramapimod, RO 30201195, SCIO 323, TACE inhibitors such as DPC 333, ICE inhibitors such as pranalcasan, and IMPDH inhibitors such as mycophenolate and merimepodib.

In combination therapy, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

The compound in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

Desirably, the methods, compositions, and kits of the invention are more effective than other methods, compositions, and kits. By “more effective” is meant that a method, composition, or kit exhibits greater efficacy, is less toxic, safer, more convenient, better tolerated, or less expensive, or provides more treatment satisfaction than another method, composition, or kit with which it is being compared.

Osteoarthritis

The methods, compositions, and kits of the invention may be used for the treatment of osteoarthritis, or pain associated therewith. If desired, one or more agents typically used to treat osteoarthritis may be used as a substitute for or in addition to a corticosteroid in the methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), NsIDIs (e.g., cyclosporine, tacrolimus, pimecrolimus, and ISAtx247), or analogs thereof. Thus, in one embodiment, the invention features the combination of an adenosine activity upregulator with any of the foregoing agents, and methods and kits for the treatment of osteoarthritis or pain associated therewith.

Chronic Obstructive Pulmonary Disease

In one embodiment, the methods, compositions, and kits of the invention are used for the treatment of chronic obstructive pulmonary disease (COPD). If desired, one or more agents typically used to treat COPD may be used as a substitute for or in addition to a corticosteroid in the methods, compositions, and kits of the invention. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline. Thus, in one embodiment, the invention features the combination of an adenosine activity upregulator and a bronchodilator, and methods of treating COPD therewith.

Psoriasis

The methods, compositions, and kits of the invention may be used for the treatment of psoriasis. If desired, one or more antipsoriatic agents typically used to treat psoriasis may be used as a substitute for or in addition to a corticosteroid in the methods, compositions, and kits of the invention. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, the invention features the combination of an adenosine activity upregulator and an antipsoriatic agent, and methods of treating psoriasis therewith.

Inflammatory Bowel Disease

The methods, compositions, and kits of the invention may be used for the treatment of inflammatory bowel disease. If desired, one or more agents typically used to treat inflammatory bowel disease may be used as a substitute for or in addition to a corticosteroid in the methods, compositions, and kits of the invention. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, the invention features the combination of an adenosine activity upregulator and any of the foregoing agents, and methods of treating inflammatory bowel disease therewith.

Rheumatoid Arthritis

The methods, compositions, and kits of the invention may be used for the treatment of rheumatoid arthritis. If desired, one or more agents typically used to treat rheumatoid arthritis may be used as a substitute for or in addition to a corticosteroid in the methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, the invention features the combination of an adenosine activity upregulator with any of the foregoing agents, and methods of treating rheumatoid arthritis therewith.

Asthma

The methods, compositions, and kits of the invention may be used for the treatment of asthma. If desired, one or more agents typically used to treat asthma may be used as a substitute for or in addition to a corticosteroid in the methods, compositions, and kits of the invention. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, the invention features the combination of an adenosine activity upregulator and any of the foregoing agents, and methods of treating asthma therewith.

Pain

The methods, compositions, and kits of the invention may be used for the treatment of pain (e.g., neuropathic pain or nociceptive pain). If desired, one or more agents typically used to treat pain may be used as a substitute for or in addition to a corticosteroid in the methods, compositions, and kits of the invention. Such agents include NSAIDs, opioids, tricyclic antidepressants, anticonvulsants, amantadine, tramadol, oxycodone, buproprion, mexiletine, and capsaicin. Thus, in one embodiment, the invention features the combination of an adenosine activity upregulator and any of the foregoing agents, and methods of treating pain therewith.

Formulation of Compositions

The administration of a combination of the invention may be by any suitable means that results in suppression of proinflammatory cytokine levels at the target region. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of the combination may be formulated in a variety of ways that are known in the art. For example, the first and second agents may be formulated together or separately. Desirably, the first and second agents are formulated together for the simultaneous or near simultaneous administration of the agents.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

The two compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Thus, for compositions adapted for oral use, an oral vehicle (e.g., a capsule) containing from between 0.01% to 25% (w/w) or more of an adenosine activity upregulator or analog and/or additional agent, preferably from between 0.01% to 10% (w/w), more preferably from between 0.05% to 4% (w/w) active agent. The capsule can be taken one to four times daily, or as needed.

Performing the methods described herein, the oral vehicle containing a adenosine activity upregulator and/or the additional agent is preferably taken orally. For example, a capsule may be taken in the morning and one in the evening by a subject suffering from an immunoinflammatory disorder or an immunoinflammatory-related disorder, like anti-platelet aggregatory activity.

Topical Formulations

Compositions can also be adapted for topical use with a topical vehicle containing from between 0.0001% and 25% (w/w) or more of the adenosine activity upregulator and between 0.001% and 25% (w/w) and more of a corticosteroid.

In a preferred combination, the corticosteroid and adenosine activity upregulator are preferably from between 0.0001% to 10% (w/w), more preferably from between 0.0005% to 4% (w/w) active agent. The cream can be applied one to four times daily, or as needed. For example, for prednisolone adapted for topical administration, a topical vehicle will contain from between 0.01% to 5% (w/w), preferably from between 0.01% to 2% (w/w), more preferably from between 0.01% to 1% (w/w) prednisolone in combination with an adenosine activity upregulator which is 0.0001% to 2% (w/w), more preferably from between 0.0005% to 1% (w/w).

Performing the methods described herein, the topical vehicle containing an adenosine activity upregulator and a corticosteroid is preferably applied to the site of discomfort on the subject. For example, a cream may be applied to the hands of a subject suffering from arthritic fingers, while topical eye drops may be applied to an eye of a subject to treat uveitis.

Inhalation

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

Dosages

Given the enhanced potency of the combinations of the invention, it is understood that a low dosage (as defined herein) of the adenosine activity upregulator and/or the additional agents can be used. These dosages will vary depending on the health and condition of the patient. Thus, a moderate dosage or even a high dosage of one or both agents can be used.

Administration of each drug in the combination can, independently, be one to four times daily for one day to one year, and may even be for the life of the patient. Chronic, long-term administration will be indicated in many cases.

The combinations of the invention are useful tools in elucidating mechanistic information about the biological pathways involved in inflammation or novel targets. Such information can lead to the development of new combinations or single agents for inhibiting proinflammatory cytokine secretion. Methods known in the art to determine biological pathways can be used to determine the pathway, or network of pathways affected by contacting cells stimulated to produce proinflammatory cytokines with the compounds of the invention.

Such methods can include, analyzing cellular constituents that are expressed or repressed after contact with the compounds of the invention as compared to untreated, positive or negative control compounds, and/or new single agents and combinations, or analyzing some other metabolic activity of the cell such as enzyme activity, nutrient uptake, and proliferation. Cellular components analyzed can include gene transcripts, and protein expression. Suitable methods can include standard biochemistry techniques, radiolabeling the compounds of the invention (e, g., ¹⁴C or ³H labeling), and observing the compounds binding to proteins, e.g. using 2d gels, gene expression profiling. Once identified, such compounds can be used in in vivo models to further validate the tool or develop new anti-inflammatory agents.

Pain and Function Indices

In order to measure the efficacy of any of the methods, compositions, or kits of the invention, a measurement index may be used. Indices that are useful in the methods, compositions, and kits of the invention for the measurement of pain associated with musculoskeletal or immunoinflammatory disorders include a visual analog scale (VAS), a Likert scale, the Lequesne index, the WOMAC index, and the AUSCAN index, each of which is well known in the art. Such indices may be used to measure pain, function, stiffness, or other variables.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain by choosing the spot on the line that best corresponds to the sensation of pain, where one end of the line corresponds to “no pain” (score of 0 cm) and the other end of the line corresponds to “unbearable pain” (score of 10 cm). This procedure provides a simple and rapid approach to obtaining quantitative information about how the patient is experiencing pain. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales and their use are described, e.g., in U.S. Pat. Nos. 6,623,040 and 6,766,319.

The Lequesne index and the Western Ontario and McMaster Universities (WOMAC) osteoarthritis index assess pain, function, and stiffness in the knee and hip of OA patients using self-administered questionnaires. Both knee and hip are encompassed by the WOMAC, whereas there is one Lequesne questionnaire for the knee and a separate one for the hip. These questionnaires are useful because they contain more information content in comparison with VAS or Likert. Both the WOMAC index and the Lequesne index questionnaires have been extensively validated in OA, including in surgical settings (e.g., knee and hip arthroplasty). Their metric characteristics do not differ significantly. The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

Indices that are useful in the methods, compositions, and kits of the invention for the measurement of pain include the Pain Descriptor Scale (PDS), the Visual Analog Scale (VAS), the Verbal Descriptor Scales (VDS), the Numeric Pain Intesity Scale (NPIS), the Neuropathic Pain Scale (NPS), the Neuropathic Pain Symptom Inventory (NPSI), the Present Pain Inventory (PPI), the Geriatric Pain Measure (GPM), the McGill Pain Questionaire (MPQ), the Short-Form McGill Pain Questionaire, the Minnesota Multiphasic Personality Inventory, the Pain Profile and Multidimensional Pain Inventory, the Child Heath Questionaire, and the Child Assessment Questionaire.

Experimental Results

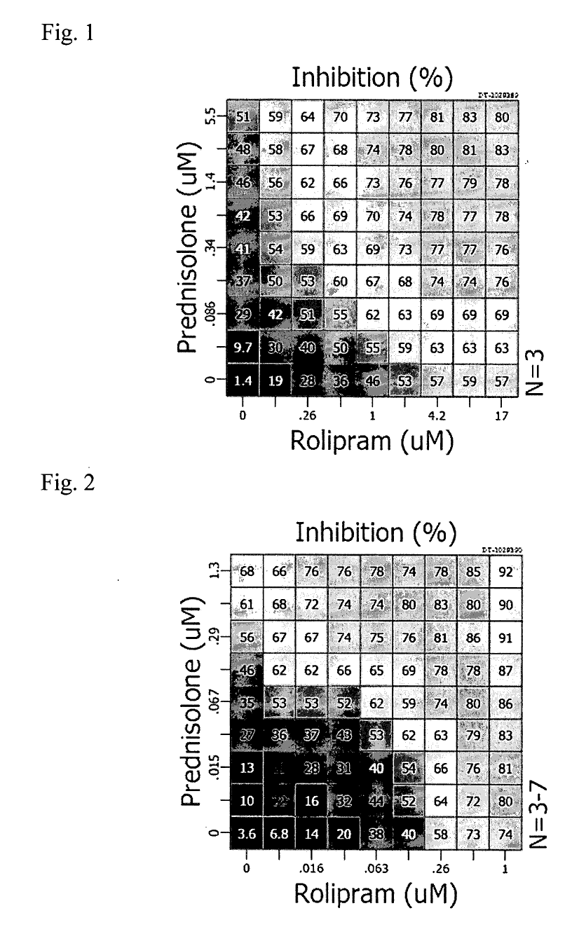

The effects of test compound combinations on TNFα secretion were assayed in white blood cells from human buffy coat stimulated with lipopolysaccharide or phorbol 12-myristate 13-acetate (PMA) and ionomycin as follows. The results from these experiments are set forth in FIGS. 1 and 2.

Lipopolysaccharide (LPS)

A 100 μl suspension of diluted human white blood cells contained within each well of a polystyrene 384-well plate (NalgeNunc) was stimulated to secrete TNFα by treatment with a final concentration of 2 μg/mL lipopolysaccharide (Sigma L-4130). Various concentrations of each test compound were added at the time of stimulation. After 16-18 hours of incubation at 37° C. in a humidified incubator, the plate was centrifuged and the supernatant transferred to a white opaque polystyrene 384-well plate (NalgeNunc, Maxisorb) coated with an anti-TNFα antibody (PharMingen, #551220). After a two-hour incubation, the plate washed (Tecan PowerWasher 384) with PBS containing 0.1% Tween 20 and incubated for an additional one hour with another anti-TNFα antibody that was biotin labeled (PharMingen, #554511) and HRP coupled to strepavidin (PharMingen, #13047E). After the plate washed with 0.1% Tween 20/PBS, an HRP-luminescent substrate was added to each well and light intensity measured using a LJL Analyst plate luminometer.

PMA/Ionomycin

A 100 μl suspension of diluted human white blood cells contained within each well of a polystyrene 384-well plate (NalgeNunc) was stimulated to secrete TNFα by treatment with a final concentration of 10 ng/mL phorbol 12-myristate 13-acetate (Sigma, P-1585) and 750 ng/mL ionomycin (Sigma, 1-0634). Various concentrations of each test compound were added at the time of stimulation. After 16-18 hours of incubation at 37° C. in a humidified incubator, the plate was centrifuged and the supernatant transferred to a white opaque polystyrene 384-well plate (NalgeNunc, Maxisorb) coated with an anti-TNFα antibody (PharMingen, #551220). After a two-hour incubation, the plate was washed (Tecan PowerWasher 384) with PBS containing 0.1% Tween 20 and incubated for an additional one hour with another anti-TNFα antibody that was biotin labeled (PharMingen, #554511) and HRP coupled to strepavidin (PharMingen, # 13047E). After the plate washed with 0.1% Tween 20/PBS, an HRP-luminescent substrate was added to each well and light intensity measured using a LJL Analyst plate luminometer.

The synergy scores calculated for the combinations of compounds set forth in FIGS. 1 and 2 was calculated by the formula S=log f_{X} log f_{Y}ΣI_{data}(I_{data}−I_{Loewe}), summed over all non-single-agent concentration pairs, and where logf_{X,Y} are the natural logarithm of the dilution factors used for each single agent. This effectively calculates a volume between the measured and Loewe additive response surfaces, weighted towards high inhibition and corrected for varying dilution factors. The synergy score indicates that the combination of the two agents provides greater inhibition of TNFα secretion than would be expected based on the activity of each agent of the combination individually. The synergy score calculated for the experiment set forth in FIG. 1 was 2.3. The synergy score calculated for the experimental results set forth in FIG. 2 was 3.7.

### Other Embodiments

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

What is claimed is:

1. A method for treating an immunoinflammatory disorder, said method comprising administering to a patient diagnosed with or at risk of developing said immunoinflammatory disorder a Group B adenosine activity upregulator and a corticosteroid simultaneously or within fourteen days of each other in amounts sufficient to treat said patient.

2. The method of claim 1, said method further comprising administering to said patient a third drug selected from the group consisting of corticosteroids; non-steroidal anti-inflammatory drugs (NSAIDs), non-steroidal immunophilin dependant immunosuppressants (NsIDI), COX-2 inhibitors; biologics; small molecule immunomodulators; DMARDs; xanthines; anticholinergic compounds; beta receptor agonists; bronchodilators; vitamin D analogs; psoralens; retinoids; and 5-amino salicylic acids, wherein said Group B adenosine activity upregulator, said corticosteroid, and said third drug are administered simultaneously or within fourteen days of each other in amounts sufficient to treat said patient.

3. The method of claim 1, wherein said immunoinflammatory disorder is rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, cirrhosis, or psoriatic arthritis.

4. The method of claim 1, wherein said Group B adenosine activity upregulator and said corticosteroid are administered within five days of each other.

5. The method of claim 4, wherein said Group B adenosine activity upregulator and said corticosteroid are administered within one day of each other.

6. The method of claim 5, wherein said Group B adenosine activity upregulator and said corticosteroid are administered within 1 hour of each other.

7. The method of claim 6, wherein said Group B adenosine activity upregulator and said corticosteroid are administered simultaneously.

8. The method of claim 1, wherein said Group B adenosine activity upregulator is adenosine, an adenosine receptor agonist, an adenosine transport inhibitor, an adenosine kinase inhibitor, an adenylate cyclase stimulant, an adenosine deaminase inhibitor, a calmodulin antagonist, or a phosphodiesterase inhibitor.

9. The method of claim 1, wherein said corticosteroid is algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin.

10. The method of claim 9, wherein said corticosteroid is prednisolone.

11. The method of claim 1, wherein said Group B adenosine activity upregulator and said corticosteroid are administered in the same pharmaceutical formulation.

12. The method of 1, wherein said Group B adenosine activity upregulator or said corticosteroid is formulated for topical administration.

13. The method of claim 1, wherein said Group B adenosine activity upregulator or said corticosteroid is formulated for systemic administration.

14. The method of claim 1, wherein said Group B adenosine activity upregulator or said corticosteroid is administered in a low dosage.

15. The method of claim 1, wherein said Group B adenosine activity upregulator or said corticosteroid is administered in a high dosage.

16. A kit comprising:
(i) a composition comprising a Group B adenosine activity upregulator and a corticosteroid, and
(ii) instructions for administering said composition to a patient diagnosed with or at risk of developing a an immunoinflammatory disorder.

17. A kit comprising:
(i) a Group B adenosine activity upregulator,
(ii) a corticosteroid, and
(iii) instructions for administering said Group B adenosine activity upregulator and said corticosteroid to a patient diagnosed with or at risk of developing an immunoinflammatory disorder.

18. A kit comprising:
(i) a corticosteroid, and
(ii) instructions for administering said corticosteroid and a Group B adenosine activity upregulator to a patient diagnosed with or at risk of developing an immunoinflammatory disorder.

19. A kit comprising:
(i) a Group B adenosine activity upregulator; and
(ii) instructions for administering said Group B adenosine activity upregulator and a corticosteroids to a patient diagnosed with or at risk of developing an immunoinflammatory disorder.
